(19) United States
(12) Patent Application Publication
(10) Pub. No.: US 2026/0256979 A1
(43) Pub. Date: Sep. 3, 2026

(54) WOUND DRESSING COMPRISING A PH SENSOR COMPOSITION AND METHOD OF PRODUCING THE SAME

(71) Applicants: Catarina Maria MARQUES DIAS, Pontével (PT); Ana Filipa BEM NUNES, Peniche (PT); Guilherme LUCAS ALVES, Guarda (PT); INSTITUTO POLITÉCNICO DA GUARDA, Guarda (PT)

(72) Inventors: Catarina Maria MARQUES DIAS, Pontével (PT); Ana Filipa BEM NUNES, Peniche (PT); Guilherme LUCAS ALVES, Guarda (PT); Sónia Alexandra PEREIRA MIGUEL, Covilhã (PT); Carla Sandra MOTA CASTRO, Viseu (PT)

(21) Appl. No.: 18/869,625

(22) PCT Filed: May 30, 2023
(86) PCT No.: PCT/IB2023/055543

§ 371 Date: Nov. 26, 2024

(30) Foreign Application Priority Data
May 31, 2022 (PT) 118027

(51) Int. Cl. A61L 15/42; A61L 15/18; A61L 15/20; A61L 15/28; A61L 15/32; A61L 15/56; A61L 15/58

(52) U.S. Cl. CPC A61L 15/425; A61L 15/18; A61L 15/20; A61L 15/28; A61L 15/32; A61L 15/56; A61L 15/585; A61L 2300/44

(57) ABSTRACT

A wound dressing comprising a porous structure, itself comprising a pH sensor composition, which is arranged on top of an adhesive film structure. The wound dressing changes colour according to pH variations at the wound site, allowing to promote the wound healing process, as well as providing proper information to health care professionals by monitoring the healing process of skin lesions, thus leading to a better care of the patient as well as the use of a more sustainable treatment. Also, disclosing a method to obtain the wound dressing.

### TECHNICAL FIELD

The present patent application discloses a wound dressing that monitors the wound healing process during the treatment of skin lesions.

### BACKGROUND ART

Skin injuries represents a critical problem for the healthcare community. Indeed, the incidences of skin injuries, namely incisional wounds (affecting 10 million people) and chronic wounds (affecting 6.7 million people), is expected to increase due to an aging population and increasing rates of diseases and conditions such as diabetes, obesity and the late effects of radiation therapy. Such will difficult more and more the management of costs and resources within the healthcare institutions.

Nowadays, different types of wound dressings have been developed which are able to minimize the occurrence of skin infections, propel the cell activity and consequently promoting the healing process. So, different biomaterials (such as hydrogels, membranes, films, etc.) have been explored due to their ability for mimicking the structure of the extracellular matrix, allowing the migration, adhesion and cell proliferation.

Document KR101429455 (B1) refers to a hydrogel bandage with antioxidant properties for treating wounds and its production method (chitosan self-assembly). More specifically, a bandage to treat wounds consisting of a mixture of polymeric hydrogel and chitosan, using an enzyme/chemical technique to transform water-insoluble chitosan into low molecular weight molecules. The present invention differs from this one in that it presents a natural film produced by a simple method, without recourse to enzyme action. Additionally, the present invention can change the colour according to the pH values.

Document CA2712527 (C) discloses a pharmaceutical composition designed to act as a protective film on the skin, composed of chitosan and agents that confer healing, calming and moisturising properties. The present invention differs from this in that the wound dressing is composed of a chitosan film and an alginate porous structure incorporating a pH sensor.

Document WO2016166731A1 discloses a wound dressing integrating reusable electronics for wireless monitoring of healing process. This device can collect and send information about different parameters such as irregular bleeding, variations in pH levels and/or blood sugar levels, and external pressure at wound site. The present invention differs from this in that it possesses a biocompatible pH sensor that indicate the pH levels at wound site through simple colour change.

Document U.S. Pat. No. 8,784,773B2 refers to hydrogel nano-composite wound dressing with an antibiotic, able to deliver at controllable manner, avoiding the microbial infections. The present invention differs from this in that the wound dressing is comprised of natural bioactive polymers that propel the healing process and, simultaneously indicate information about wound healing progression.

Document US20160015962A1 refers to a flexible patch incorporating different sensors that monitor signs of infection, as well as eliminate bacterial infections through electrical stimulation. The present invention differs from this in that the pH sensor used is non-cytotoxic and cost-effective and non-requiring complicated process to monitor the healing process.

It is also important to highlight the aspects that stand out in the present invention in relation to the research works available in literature. The work entitled “Development of a new chitosan hydrogel for wound dressing” (Ribeiro et al., 2009) describes a hydrogel only composed of chitosan that requires periodic changes during the treatment. The present invention differs in that the proposed wound dressing is composed of a film and central porous material incorporating a pH sensor which alters it colour according to the pH values at wound site.

The work entitled “Thermoresponsive chitosan-agarose hydrogel for skin regeneration” (Miguel et al., 2014) describes a thermoresponsive biocompatible and antibacterial hydrogel capable of promoting wound healing. The present invention differs from this system since the chitosan is one of the compounds of the film, and also there is another alginate material incorporating a pH sensor.

The work entitled “Electrospun Polycaprolactone/Aloe Vera_chitosan Nanofibrous Asymmetric Membranes Aimed for Wound Healing Applications” (Miguel et al., 2017) describes the production of an asymmetric membrane through the process of electrospinning. The present invention differs from this one in that the dressing composed of a film and materials that possess exceptional biological properties as well as is able to monitor the healing process status.

The work entitled “An Integrated Smart Sensor Dressing for Real-Time Wound Microenvironment Monitoring and Promoting Angiogenesis and Wound Healing” (Zhang et al., 2021) describes the production of UV-crosslinked hydrogel incorporating microenvironment sensor connected to an antenna capable to receive, display and upload wound microenvironment data in real time. The present invention differs from this in that the dressing is able to monitor the wound healing process through the simple alteration of its colour.

The work entitled “Smart Dual-Sensor Wound Dressing for Monitoring Cutaneous Wounds” (Mirani et al., 2023) describes the incorporation of colorimetric glucose sensors and antibiotics/growth factor-releasing modulus into gels, aims to monitor and treat diabetic chronic wounds. The present invention differs from this in that the dressing incorporates a pH biological dye that monitor the pH alterations through the change of colour.

The work entitled “Smart wound dressing for infection monitoring and NIR-triggered antibacterial treatment” (Qiao et al., 2020) describes the production and characterization of a smart hydrogel-based wound dressing capable of monitoring bacterial via infection a pH-responsive fluorescence resonance energy transfer (FRET) transition of Cyanine3 (Cy3) and Cyanine5 (Cy5) in a bacterial environment and providing on-demand treatment of infection via near infrared (NIR) light-triggered antibiotic release. The present invention differs from this in that the dressing is able to inform about the occurrence of a skin infection, since it changes the colour to pink due to increase of pH values at wound site.

However, the periodical replacement of wound dressings constitutes a task that involve high costs, time of professional and sometimes such procedure is done without need. So, in these situations, the wound dressing is discarded, and new dressing is placed on wound site. Moreover, the wound dressing substitution can induce pain and discomfort to the patient and increase the risk of skin infections.

### SUMMARY

The present application relates to a wound dressing comprising a porous structure arranged on an adhesive film structure, wherein:
the porous structure comprises alginate in a concentration between 1% and 2% (w/v), a pH sensor composition in a concentration between 0.5% and 1% (w/v); and
the adhesive film structure comprises chitosan in a concentration between 0.5 and 1% (w/v), hyaluronic acid in a concentration between 1 and 2% (w/v), a thickening agent in a concentration between 0.05 and 0.20% (w/v), and a preservation agent between 0.05 and 0.10% (w/v).

In one embodiment, the pH sensor composition is selected from phenol red, rosolic acid, benzophenone azo, thymolphthalein, or curcumin.

In one embodiment, the wound dressing further comprises other compounds selected from cellulose acetate, gelatin, agarose, or fucoidan.

In one embodiment, the thickening agent is selected from ethylene glycol, mannitol, xylitol, glycerine, polysaccharides such as starches, vegetable gums, or pectin, proteins such as collagen or gelatin, or mixtures thereof.

In one embodiment, the preservation agent is selected from benzoic acid, sodium benzoate, sorbic acid, sodium sorbate, parabens, isothiazolinones, benzyl alcohol or mixtures thereof.

The present application also relates to method to obtain the wound dressing comprising the following steps:
Preparing a porous structure by:
   dissolving alginate between 1 and 2% wt. in 100 ml of water;
   adding a pH sensor composition between 0.5 and 1% (w/v) to the previous solution and homogenizing;
   placing the mixture in a mould for wound dressing with a pre-defined size;
   adding a solution comprising divalent ions in a concentration between 0.5 and 2% (w/v) to the mixture;
   freeze-drying the mixture to obtain the porous structure;
Preparing an adhesive film structure by:
   dissolving chitosan between 0.5 and 1% wt. in 100 mL of an acidic solution between 0.5 and 1% v/v;
   adding a hyaluronic acid solution between 1 and 2% (w/v) to the previous mixture;
   adding a thickening agent between 0.05 and 0.20% (w/v);
   adding a preservation agent between 0.05 and 0.10% (w/v);
   homogenizing the mixture avoiding air incorporation;
   adjusting the pH of the mixture between 5.0 and 7.4;
   integrating the porous structure previously prepared in a mould for wound dressing with a pre-defined size;
   adding the adhesive film solution into the mould allowing the porous structure to be centred in the mould;
   allowing the solvent to evaporate until the wound dressing is obtained.

In one embodiment, the pH sensor composition is selected from phenol red, rosolic acid, benzophenone azo, thymolphthalein, or curcumin.

In one embodiment, the solution comprising divalent ions is selected from calcium chloride, or solutions comprising magnesium or iron ions.

In one embodiment, the acidic solution to dissolve chitosan is selected from lactic acid, acetic acid, or hydrochloric acid.

In one embodiment, the mould size for the porous structure is selected from 4×4 cm, 2×8 cm or 4×10 cm.

In one embodiment, the mould size for the adhesive film structure is selected from 8×8 cm, 4×16 cm or 8×20 cm.

In one embodiment, the thickening agent is selected from ethylene glycol, mannitol, xylitol, glycerine, polysaccharides such as starches, vegetable gums, or pectin, proteins such as collagen or gelatin, or mixtures thereof.

In one embodiment, the preservation agent is selected from benzoic acid, sodium benzoate, sorbic acid, sodium sorbate, parabens, isothiazolinones, benzyl mixtures thereof.

### GENERAL DESCRIPTION

The present invention is based on the development of a skin wound dressing incorporated with a pH sensor which allows the monitoring of the healing process.

This wound dressing, in addition to having the capacity to confer a moist wound environment and having a qualitative constitution based on products, natural will also incorporate a pH sensor which will promote the change of the colour of the dressing according to that variation. In this way, if the pH of the wound bed increases and there are pH values >7, indicating a basic character, the wound dressing changes colour from original red colour to pink (when using phenol red) and indicates that dressing replacement is required, as there may be a significant amount of exudate present in the wound bed, which will hinder the normal healing process. On contrary, if the pH of the wound bed decreases to pH<7 values, the wound dressing will acquire a yellow color (when using phenol red), signaling the wound regeneration process is happening.

The composition of the wound dressing disclosed herein stands out from those on the market in that it has a qualitative natural product formulation and is able to monitor and signal, through colour change, not requiring the need for dressing change.

The present invention, which is an adhesive film-based wound dressing, comprises chitosan and hyaluronic acid in its composition, which will create a protective physical barrier between the lesion site and the external environment.

In addition, the structure of the wound dressing comprises a porous, permeable structure which will incorporate the pH sensor. A pH indicator can be selected from those widely used in cell biology, which substantiates its applicability in biomedical applications.

This invention has been developed in order to present a qualitative bioactive composition capable of promoting the wound healing process, as well as functioning as a wound dressing that indicates to the health professional, to the primary care provider, or to the patient, the need for dressing replacement. In this way, it is possible to avoid periodic changes of wound dressings, which have several disadvantages such as costs, cause pain and discomfort for the patient, at the time of replacement may remove tissue that is forming and increase the risk of bacterial contamination, and in this way also make life easier for health professionals.

### BRIEF DESCRIPTION OF DRAWINGS

For easier understanding of this application, figures are attached in the annex that represent the preferred forms of implementation which nevertheless are not intended to limit the technique disclosed herein.

FIG. 1 shows a top view of the wound dressing of the present application.

FIG. 2 shows a side view of the wound dressing of the present application.

FIG. 3 shows the swelling profile of the porous structures and adhesive film structures when immersed in PBS solution (pH 5.0).

FIG. 4 shows the total porosity of the porous structures.

### DETAILED DESCRIPTION OF EMBODIMENTS

Now, preferred embodiments of the present application will be described in detail with reference to the annexed drawings. However, they are not intended to limit the scope of this application.

The present invention relates to the development of a wound dressing, based on components of natural origin for wound dressing, incorporated with a pH sensor.

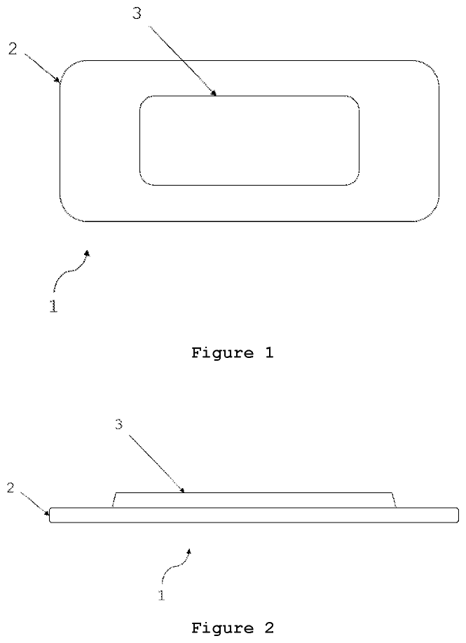

As shown in FIGS. 1 and 2, the wound dressing (1) herein described comprises a porous structure (3), itself comprising a pH sensor composition, which is arranged on an adhesive film structure (2).

In one embodiment, the porous structure is integrated in the central region of the adhesive film structure.

In one embodiment the porous structure comprises alginate at a concentration that can vary between 1% and 2% (w/v), which will assure the wound exudate absorption, conferring a moist environment at wound site.

In one embodiment, the pH sensor composition comprises a pH indicator selected from, but not limited to, phenol red, rosolic acid, benzophenone azo, thymolphthalein, or curcumin.

In a preferred embodiment, the pH sensor composition is phenol red.

Phenol red is a pH indicator widely used in cell biology, which underlies its applicability in biomedical applications (Paul Held, USA 2021). Also, this dye is non-cytotoxic and cost-effective, overcoming the complex design involved and the costs associated to the fabrication and integration of biosensors into wound dressings.

In one embodiment the pH sensor composition concentration is between 0.5% and 1% (w/v), that confer colour to the porous structure.

In one embodiment the adhesive film structure comprises between 0.5 and 1% (w/v) of chitosan, that assures the adhesiveness, flexibility as well as antibacterial properties to the film.

In one embodiment the adhesive film structure further comprises hyaluronic acid ranging between 1 and 2% (w/v), providing bioactive properties to the wound dressing, which will encourage the cell activity, and hence the formation of the new tissue.

The presence of chitosan and hyaluronic acid allows to create a physical barrier of protection between the site of the injury and the external environment as well as confer the hydrophilic, bioactive, antibacterial properties to the dressing.

The presence of alginate allows to create a sponge able to absorb the excess of wound exudate, providing a moist environment at wound site suitable for skin regeneration process.

The adhesive film structure further comprises a thickening agent between 0.05 and 0.20% (w/v) selected from, but not limited to, ethylene glycol, mannitol, xylitol, glycerine, polysaccharides such as starches, vegetable gums, or pectin, proteins such as collagen or gelatin, or mixtures thereof.

The adhesive film structure further comprises a preservation agent between 0.05 and 0.10% (w/v) selected from, but not limited to, benzoic acid, sodium benzoate, sorbic acid, sodium sorbate, parabens, isothiazolinones, benzyl alcohol or mixtures thereof.

As optional compounds, the porous structure or the adhesive film structure of the wound dressing can comprise other natural biological constituents namely, but not limited to, cellulose acetate, gelatin, agarose, or fucoidan, among others. The selection of the natural polymers will assure the biocompatibility of the system, that is considered a crucial parameter for biomedical applications.

The wound dressing (1) is obtained according to a method comprising the following steps:
Preparing a porous structure (3) by:
   dissolving alginate between 1 and 2% wt. in 100 ml of water;
   adding a pH sensor composition between 0.5 and 1% (w/v) to the previous solution and homogenizing;
   placing the mixture in a mould for wound dressing with a pre-defined size;
   adding a solution comprising divalent ions in a concentration between 0.5 and 2% (w/v) to the mixture;
   freeze-drying the mixture to obtain the porous structure;
Preparing an adhesive film structure (2) by:
   dissolving chitosan between 0.5 and 1% wt. in 100 mL of an acidic solution between 0.5 and 1% v/v;
   adding a hyaluronic acid solution between 1 and 2% (w/v) to the previous mixture;
   adding a thickening agent between 0.05 and 0.20% (w/v);
   adding a preservation agent between 0.05 and 0.10% (w/v);
   homogenizing the mixture avoiding air incorporation;
   adjusting the pH of the mixture between 5.0 and 7.4;
   integrating the porous structure previously prepared in a mould for wound dressing with a pre-defined size;
   adding the adhesive film solution into the mould allowing the porous structure to be centred in the mould;
   allowing the solvent to evaporate until the wound dressing (1) is obtained.

In one embodiment, the pH sensor composition comprises a pH indicator selected from, but not limited to, phenol red, rosolic acid, benzophenone azo, thymolphthalein, or curcumin.

In one embodiment, the solution comprising divalent ions is selected from, but not limited to, calcium chloride, or solutions comprising magnesium or iron ions.

In one embodiment, the acidic solution to dissolve chitosan is selected from, but not limited to, lactic acid, acetic acid, or hydrochloric acid.

In one embodiment, the pH of the mixture is adjusted with a sodium hydroxide solution, sodium carbonate solution, ammonium hydroxide solution, calcium hydroxide solution or magnesium hydroxide solution.

In one embodiment, the mould size for the porous structure is selected from 4×4 cm, 2×8 cm or 4×10 cm.

In one embodiment, the mould size for the adhesive film structure is selected from 8×8 cm, 4×16 cm or 8×20 cm.

In one embodiment, the thickening agent is selected from, but not limited to, ethylene glycol, mannitol, xylitol, glycerine, polysaccharides such as starches, vegetable gums, or pectin, proteins such as collagen or gelatin, or mixtures thereof.

In one embodiment, the preservation agent is selected from, but not limited to, benzoic acid, sodium benzoate, sorbic acid, sodium sorbate, parabens, isothiazolinones, benzyl alcohol or mixtures thereof.

The solution comprising divalent ions acts as crosslinker agent, assuring the stability of the alginate porous structure of the dressing, after wound exudate absorption.

The alginate is a natural polysaccharide that assure the hydrophilicity of the central porous structure required to absorb the wound exudate and simultaneously provide moist environment at wound site. Alginate has a great potential for the synthesis of sponges with high capacity to absorb exudate characteristic of wound, also having homeostatic properties, reducing the probability of infection development.

The phenol red is a pH dye able to change the colour according to the pH value at wound site. Through the incorporation of the phenol red into alginate porous matrix will inform about the pH alterations along the progression of healing process. So, the initial red colour will change to yellow if the pH decrease (indicating that the wound healing process occurs normally without complications) and change to pink if the pH value increases (characteristic of the presence of bacterial growth). Such information will enable to monitor the healing process, without the dressing removal.

Chitosan is a natural polysaccharide that possess auspicious properties for biomedical applications namely, it confers adhesiveness, antibacterial properties to the film. Also, chitosan will encourage the cell proliferation. It has high activity during the diverse phases of wound healing: in homeostasis the compound has the capacity to promote platelet adhesion and aggregation, helping the clot synthesis. During inflammatory phase, the polymer has an antimicrobial profile that allows to eliminate every bacteria causing infection, assisting also inflammatory cells responsible for cleaning the death tissue. Nevertheless, in proliferative phase chitosan interfere with the synthesis of cytokines crucial for wound healing process.

Hyaluronic acid, as the component of extracellular matrix of the native skin, will confer bioactivity and hydrophilicity adequate for cell activity. Hyaluronic acid (include in a glycosaminoglycan family) is one the most abundant compounds of the extracellular matrix with the capacity to modulate specific processes like cell proliferation and angiogenesis, crucial during the tissue regeneration.

Thickening agents will increase the viscosity of solutions, improving the structural integrity of the films.

The preservation agents s will prevent decomposition by microbial growth or by undesirable chemical changes.

Some results referring to the swelling profile and porosity are presented in following images.

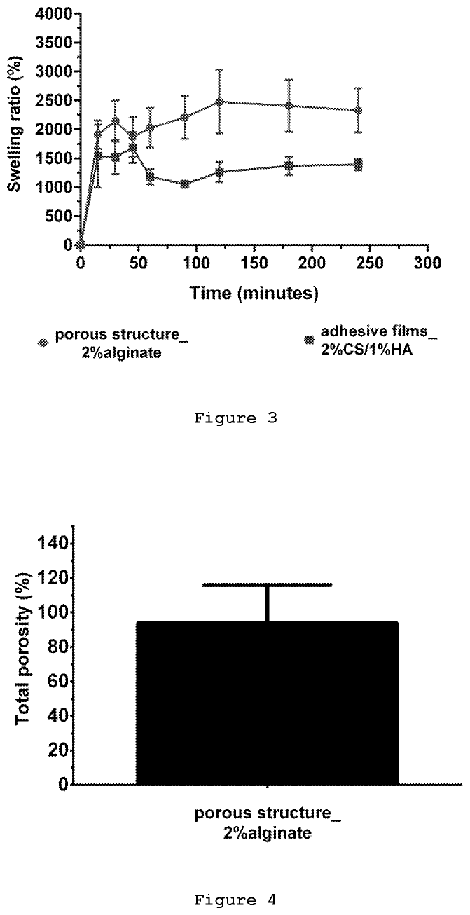

In FIG. 3, it is presented the swelling ratio of porous structures and adhesive films, when immersed in PBS solution at pH 5.5.

In FIG. 4, it is presented the total porosity of the porous structures.

It is clearly evident the high ability of the porous structure for water absorption in comparison to the adhesive film structure. In the present invention, this result is crucial to absorb the excess of wound exudate available on chronic wounds, and consequently the pH sensor will promote the change of the colour. Also, in the formulations with higher concentrations of alginate, the swelling ratio also decreases.

In relation to the adhesive film structure, this part of the wound dressing is also capable to absorbing fluids, but after 45 minutes, the swelling ratio decreased. Such result is desirable, since the adhesive film structure is responsible for the integrity of the wound dressing, assuring the adherence to the skin. The variations between chitosan and hyaluronic acid concentrations on the adhesive film structure composition have an impact in the properties like adhesiveness and structural integrity. In terms of the swelling ratio, it is expected that the values will be similar.

According to the results obtained for the porosity of porous structure, it is visible that it has great porosity with a mean of 94%. Such property is crucial for the absorption of the exudate characteristic of the wounds and also for the availability of the pH indicator with wound exudate. However, high porosity can compromise the stability of the porous structure.

In the following tables, the different formulations that were tested for the production of the present invention are presented. These show essentially the variations between the concentration of chitosan, hyaluronic acid, alginate and phenol red. These examples helped develop an ideal formulation presenting morphological, physic-chemical, and biological properties for the envisioned application. So, the concentration variation of alginate in the porous structure has an impact on swelling, porosity, and stability of the system. In turn, chitosan and hyaluronic acid variations on the adhesive film structure composition affects its adhesiveness, integrity, and transparency. In addition, the concentration of phenol red was selected according to its capacity to detect the differences on pH values, not compromising the biocompatibility of the system.

### EXAMPLES

### Example 1

| Porous | Alginate | 2.0 | g | 2.0% |
|---|---|---|---|---|
| structure | Phenol Red | 1 | g | 1.0% |
| Adhesive | Chitosan | 1.0 | g | 1.0% |
| films | Lactic acid/Acetic Acid | pH 5.5 | | 0.5%-1% |
| | Hyaluronic acid | 2.0 | g | 2.0% |
| | Thickening agents | 0.05-0.2 | g | 0.050%-0.2% |
| | Preservation agents | 0.05-0.1 | g | 0.05%-0.1% |

The wound dressing presented a porous structure with lower swelling ratio and porosity, since the concentration of alginate was higher, promoting the formation of a more compact structure. The adhesive film structure comprising 1% of chitosan and 2% of hyaluronic acid led to higher mucoadhesiveness and optimal biological properties.

### Example 2

| Porous | Alginate | 1.5 | g | 1.5% |
|---|---|---|---|---|
| structure | Phenol Red | 1 | g | 1.0% |
| Adhesive | Chitosan | 1.0 | g | 1.0% |
| films | Lactic acid/Acetic Acid | pH 5.5 | | 0.5%-1% |
| | Hyaluronic acid | 2.0 | g | 2.0% |
| | Thickening agents | 0.05-0.2 | g | 0.05%-0.2% |
| | Preservation agents | 0.05-0.1 | g | 0.05%-0.1% |

The alginate concentration at 1.5% on the porous structure promoted a moderate swelling ratio and porosity. The adhesive film structure comprising 1% of chitosan and 2% of hyaluronic acid led to higher mucoadhesiveness and optimal biological properties.

### Example 3

| Porous | Alginate | 1.0 | g | 1.5% |
|---|---|---|---|---|
| structure | Phenol Red | 1 | g | 1.0% |
| Adhesive | Chitosan | 1.0 | g | 1.0% |
| films | Lactic acid/Acetic Acid | pH 5.5 | | 0.5%-1% |
| | Hyaluronic acid | 2.0 | g | 2.0% |
| | Thickening agents | 0.05-0.2 | g | 0.05%-0.2% |
| | Preservation agents | 0.05-0.1 | g | 0.05%-0.1% |

The alginate concentration at 1.0% on the porous structure promoted a higher swelling ratio and porosity. The adhesive film structure comprised 1% of chitosan and 2% of hyaluronic acid lead to higher mucoadhesiveness and optimal biological properties.

### Example 4

| Porous | Alginate | 2.0 | g | 2.0% |
|---|---|---|---|---|
| structure | Phenol Red | 1 | g | 1.0% |
| Adhesive | Chitosan | 0.5 | g | 0.5% |
| films | Lactic acid/Acetic Acid | pH 5.5 | | 0.5%-1% |
| | Hyaluronic acid | 2.0 | g | 2.0% |
| | Thickening agents | 0.05-0.2 | g | 0.05%-0.2% |
| | Preservation agents | 0.05-0.1 | g | 0.05%-0.1% |

The wound dressing presented a porous structure with lower swelling ratio and porosity, since the concentration of alginate was higher, promoting the formation of a more compact structure. The adhesive film structure comprised 0.5% of chitosan and 2% of hyaluronic acid leading to low mucoadhesiveness and structural integrity.

### Example 5

| Porous | Alginate | 2.0 | g | 2.0% |
|---|---|---|---|---|
| structure | Phenol Red | 1 | g | 1.0% |
| Adhesive | Chitosan | 1 | g | 1% |
| films | Lactic acid/Acetic Acid | pH 5.5 | | 0.5%-1% |
| | Hyaluronic acid | 1.0 | g | 1.0% |
| | Thickening agents | 0.05-0.2 | g | 0.05%-0.2% |
| | Preservation agents | 0.05-0.1 | g | 0.05%-0.1% |

The wound dressing presented a porous structure with lower swelling ratio and porosity, since the concentration of alginate was higher, promoting the formation of a more compact structure. The adhesive film structure comprised 1% of chitosan and 1% of hyaluronic acid led to higher mucoadhesiveness and optimal biological properties. A higher concentration of hyaluronic acid into adhesive films composition improved the tissue interaction and wettability of the wound dressing.

### Example 6

| Porous | Alginate | 2.0 | g | 2.0% |
|---|---|---|---|---|
| structure | Phenol Red | 0.5 | g | 0.5% |
| Adhesive | Chitosan | 1.0 | g | 1.0% |
| films | Lactic acid/Acetic Acid | pH 5.5 | | 0.5%-1% |
| | Hyaluronic acid | 2.0 | g | 2.0% |
| | Thickening agents | 0.05-0.2 | g | 0.05%-0.2% |
| | Preservation agents | 0.05-0.1 | g | 0.05%-0.1% |

The wound dressing presented a porous structure with lower swelling ratio and porosity, since the concentration of alginate was higher, promoting the formation of a more compact structure. The adhesive film structure comprising 1% of chitosan and 2% of hyaluronic acid led to higher mucoadhesiveness and optimal biological properties.

This description is of course not in any way restricted to the forms of implementation presented herein and any person with an average knowledge of the area can provide many possibilities for modification thereof without departing from the general idea as defined by the claims. The preferred forms of implementation described above can obviously be combined with each other. The following claims further define the preferred forms of implementation.

1. A wound dressing comprising a porous structure arranged on an adhesive film structure, wherein:
the porous structure comprises alginate in a concentration between 1% and 2% (w/v), a pH sensor composition in a concentration between 0.5% and 1% (w/v); and
the adhesive film structure comprises chitosan in a concentration between 0.5 and 1% (w/v), hyaluronic acid in a concentration between 1 and 2% (w/v), a thickening agent in a concentration between 0.05 and 0.20% (w/v), and a preservation agent between 0.05 and 0.10% (w/v).

2. The wound dressing according to claim 1, wherein the pH sensor composition is selected from the group consisting of phenol red, rosolic acid, benzophenone azo, thymolphthalein, and curcumin.

3. The wound dressing according to claim 1, further comprising compounds selected from cellulose acetate, gelatin, agarose, or fucoidan.

4. The wound dressing according to claim 1, wherein the thickening agent is selected from the group consisting of ethylene glycol, mannitol, xylitol, glycerine, polysaccharides, vegetable gums, pectin, proteins, and mixtures thereof.

5. The wound dressing according to claim 1, wherein the preservation agent is selected from the group consisting of benzoic acid, sodium benzoate, sorbic acid, sodium sorbate, parabens, isothiazolinones, benzyl alcohol and mixtures thereof.

6. A method to obtain the wound dressing described in claim 1, comprising the following steps:
preparing a porous structure by:
dissolving alginate between 1 and 2% wt. in 100 mL of water;
adding a pH sensor composition between 0.5 and 1% (w/v) to the previous solution and homogenizing;
placing the mixture in a mould for wound dressing with a pre-defined size;
adding a solution comprising divalent ions in a concentration between 0.5 and 2% (w/v) to the mixture;
freeze-drying the mixture to obtain the porous structure;
preparing an adhesive film structure by:
dissolving chitosan between 0.5 and 1% wt. in 100 mL of an acidic solution between 0.5 and 1% v/v;
adding a hyaluronic acid solution between 1 and 2% (w/v) to the previous mixture;
adding a thickening agent between 0.05 and 0.20% (w/v);
adding a preservation agent between 0.05 and 0.10% (w/v);
homogenizing the mixture avoiding air incorporation;
adjusting the pH of the mixture between 5.0 and 7.4;
integrating the porous structure previously prepared in a mould for wound dressing with a pre-defined size;
adding the adhesive film solution into the mould allowing the porous structure to be centred in the mould;
allowing the solvent to evaporate until the wound dressing is obtained.

7. The method according to claim 6, wherein the pH sensor composition is selected from the group consisting of phenol red, rosolic acid, benzophenone azo, thymolphthalein, or and curcumin.

8. The method according to claim 6, wherein the solution comprising divalent ions is selected from calcium chloride, or solutions comprising magnesium or iron ions.

9. The method according to claim 6, wherein the acidic solution to dissolve chitosan is selected from lactic acid, acetic acid, or hydrochloric acid.

10. The method according to claim 6, wherein the mould size for the porous structure is selected from 4×4 cm, 2×8 cm or 4×10 cm.

11. The method according to claim 6, wherein the mould size for the adhesive film structure is selected from 8×8 cm, 4×16 cm or 8×20 cm.

12. The method according to claim 6, wherein the thickening agent is selected from the group consisting of ethylene glycol, mannitol, xylitol, glycerine, polysaccharides, vegetable gums, pectin, proteins, and mixtures thereof.

13. The method according to claim 6, wherein the preservation agent is selected from the group consisting of benzoic acid, sodium benzoate, sorbic acid, sodium sorbate, parabens, isothiazolinones, benzyl alcohol and mixtures thereof.
